(19) United States
(12) Patent Application Publication
(10) Pub. No.: US 2014/0350106 A1
(43) Pub. Date: Nov. 27, 2014

(54) Urea Silicone Gel for Scars and Hydration Treatment and Method of Using Same

(71) Applicant: Fabiana Campanati Banov, Sugar Land, TX (US)

(72) Inventor: Fabiana Campanati Banov, Sugar Land, TX (US)

(73) Assignee: PROFESSIONAL COMPOUNDING CENTERS OF AMERICA, Houston, TX (US)

(21) Appl. No.: 13/900,360

(22) Filed: May 22, 2013

(51) Int. Cl. A61K 47/34; A61K 31/203; A61K 31/17

(52) U.S. Cl. CPC A61K 47/34; A61K 31/17; A61K 31/203
USPC 514/559; 514/588

(57) ABSTRACT

Compositions and methods for urea silicone gels for treating skin conditions that may benefit from barrier protection and from urea silicone gel ability to return water balance to the skin are disclosed. Skin conditions that may be treated with urea silicone gel may be excessive dryness, insect bites, keloids and scars, among others. Disclosed urea silicone gel may include micronized urea USP, an anhydrous silicone base, and a PEG ointment base, among other ingredients. Anhydrous silicone base may include Amazonian oils such as pracaxi oil and seje oil, which are rich in oleic, linolenic, linoleic acids, and sterols, particularly beta-sitosterol and stigmasterol that may increase skin permeability to urea. PEG ointment base may be water-washable and includes meadowsweet extract. Additionally, PEG urea silicone gel may provide occlusion, and may maintain a moist environment within the skin condition which allows optimal healing. Pain also may be decreased by maintaining a moist environment.

### BACKGROUND

1. Field of the Disclosure

The present disclosure relates to topical delivery of urea, and more specifically to permeable enhanced compositions and methods for treating skin conditions such as scars and dryness.

2. Background

Urea preparations may be used for treating dry skin, cracked skin, scars, hyperkeratotic conditions, and to soften the skin and nails, among other skin conditions. Urea formulations may break down skin cells and may speed the overall process of healing, as well as soften skin and nails.

Some topical compositions for scars may include salicylic acid, which exhibit side effects such as skin irritation, dryness, peeling, and redness. Skin irritation may occur in the form of itchiness, burning, or stinging.

Urea topical compositions do not exhibit side effects described in the previous paragraph because of urea's special ability to hydrate dry skin by drawing moisture into the cell structure of the stratum corneum. Urea may soften hard skin, attract and retain moisture and increase the penetration of other medications. Urea compositions work by breaking down hard, dead skin cells.

Scars vary greatly in quality, depending on individual and racial patient features, the nature of the trauma, and the conditions of skin condition healing. Scars frequently determine aesthetic impairment and may be symptomatic, causing itching, tenderness, pain, sleep disturbance, anxiety, depression, and disruption of daily activities. Other psychological sequelae may include post-traumatic stress reactions, loss of self-esteem and stigmatization leading to a diminished quality of life. Scar contractures also can determine disabling physical deformities. All these problems are more troublesome to the individual patient, particularly when the scar cannot be hidden by clothes.

While many treatments have been suggested, only two treatments can be said to have sufficient evidence for scar management; topical application of silicone gel sheeting and the intralesional injection of corticosteroids. The former generally is indicated as both a preventive and therapeutic agent. Topical silicone gel sheeting is cumbersome to keep on the scar, and the patient compliance often is low for lesions in visible areas. Tapes or bandaging may lead to skin irritation, which can require discontinuation of treatment, especially in hot climates. Steroid injections are painful, may lead to skin atrophy and dyschromies. Additionally, surgical removal of scars can cause a keloid to return even larger.

There is therefore a need for compositions and methods for urea gels that includes silicone gel base with natural antimicrobial, moisturizing oils that may increase skin permeability for urea and exhibit healing and anti-inflammatory properties for enhanced treatments for skin conditions, such as scars.

### SUMMARY

Compositions and methods for urea silicone gel are described. Disclosed urea silicone gel may include at least two Amazonian oils, pracaxi oil and seje oil that may enhance skin permeability to urea. Disclosed urea silicone gel may be applied on body surface in order to moisturize skin and treat skin conditions such as cracked skin, scars, and keloids, among other skin conditions. Urea silicone gel may be a safe and effective treatment for scars such as hypertrophic and keloidal scars by helping to soften and fade keloids. Urea silicone gel may dissolve the intercellular matrix of the cells of the stratum corneum, promoting desquamation of scaly skin, eventually resulting in softening of hyperkeratotic areas. Additionally, urea silicone gel helps the stratum corneum maintain its capacity to retain water, effectively stimulating skin hydration and providing long-term results. Due to Amazonian oils within urea silicone gel, disclosed urea silicone gel may also exhibit anti-inflammatories and antimicrobial properties.

In other embodiments, disclosed urea silicone gel may be employed to treat skin spots, severe acne in the body (and scars), stretch marks, and may be useful for psoriasis and rosacea.

In another aspect of the disclosure, urea silicone gel may be employed to prevent itching associated with scars or caused by insect bites and mild skin rashes.

In one embodiment, urea silicone gel may include micronized urea USP as active pharmaceutical ingredient (API), an anhydrous silicone base that includes Amazonian oils, and a polyethylene glycol (PEG) ointment base, among other ingredients.

Anhydrous silicone base within urea silicone gel may include pracaxi oil and seje oil, which are rich in oleic, linolenic, linoleic acids, and sterols, particularly beta-sitosterol and stigmasterol that may increase skin permeability to urea or any other API. In other embodiments, urea silicone gel may also include Plukenetia Volubilis seed oil, and Inaja oil, among other oils.

PEG ointment base is a soft, opaque, water-washable ointment base that includes organic meadowsweet extract. Due to the phenolic glycosides (spiraein) and flavonoids in the meadowsweet, PEG ointment base may have germicidal, anti-inflammatory and healing properties, therefore PEG ointment base may be employed to treat sores, and cuts, among other skin conditions.

In other embodiments, urea silicone gel may include a second with or without a third suitable API. Various suitable additives, known to those skilled in the art, may be included in disclosed urea silicone gel.

Suitable amount of micronized urea USP within urea silicone gel may be from about 8% by weight to about 20% by weight; most suitable amount may be from about 15% by weight to about 20% by weight. Amount of anhydrous silicone base that may be included in disclosed urea silicone gel may range from about 5% by weight to about 40% by weight; most suitable amount may be of about 10% by weight to about 25% by weight. Furthermore, amount of PEG ointment base that may be included in disclosed urea silicone gel may range from about 1% by weight to about 95% by weight; most suitable amount may be of about 10% by weight to about 20% by weight.

In one embodiment, producing urea silicone gel may be achieved by mixing ingredients of urea silicone gel in a homogenizer such as a high shear homogenizer. The method may further include dispersing urea silicone gel in a vessel employing a high shear homogenizer, dispersing urea silicone gel at speeds of about 5,000 rotations per minute (RPM).

In one embodiment, urea silicone gel may be applied to any area of skin such as face, heels, joints, and other parts of the skin.

According to an embodiment, urea silicone gel may be applied directly to the scar, twice daily (about 2 to 6 grams).

Increasing the permeability to urea, the time of treatment may be significantly reduced, therefore reducing the time of results of treatment to a period of from about 3 weeks to about 6 weeks.

Numerous other aspects, features of the present disclosure may be made apparent from the following detailed description.

### DETAILED DESCRIPTION

The present disclosure is here described in detail. Other embodiments may be used and/or and other changes may be made without departing from the spirit or scope of the present disclosure. The illustrative embodiments described in the detailed description are not meant to be limiting of the subject matter presented here.

### DEFINITIONS

As used here, the following terms may have the following definitions:

“Treating” and “Treatment” refer to reduction in severity and/or frequency of symptoms, elimination of symptoms and/or underlying cause, prevention of the occurrence of symptoms and/or their underlying cause, and improvement or remediation of damage.

“Permeation enhancement” refers to an increase in the permeability of the skin to the selected active pharmaceutical ingredient.

“Emollient” refers to a substance having the quality of softening or soothing the skin.

“Gel” refers to a colloid in which the disperse phase has combined with the dispersion medium to produce a semisolid material, such as a jelly that may include a cosmetic, medicinal, or other preparation.

“Scar” refers to a growth of collagen beneath the skin that may be formed as the result of wound healing.

“Keloids” refers to benign fibrous growths that occur after trauma or wounding of the skin and present a major therapeutic dilemma to the dermatologist because of frequent recurrences.

“Silicone” refers to polymeric organic silicon compounds obtained as oils.

### DESCRIPTION

Embodiments of the present disclosure may be directed towards compositions and methods for urea silicone gels that include Amazonian oils which may enhance skin permeability to urea or any other active pharmaceutical ingredient (API). Urea silicone gels may be applied on body surface in order to moisturize skin and treat skin conditions such as excessive skin dryness, cracked skin, stretch marks, scar tissues, and keloids, among other skin conditions. Scar tissues may include new scars, old scars, and surgical scars, among others. Urea silicone gel may exhibit healing and soothing power, and emolliency.

Composition

In one embodiment, the present disclosure provides topical formulations that may be employed for scar treatment.

Disclosed urea silicone gel may include micronized urea USP, an anhydrous silicone base, and a polyethylene glycol (PEG) ointment base, among other ingredients.

In other embodiments, urea silicone gel may not include PEG ointment base.

Active Pharmaceutical Ingredients

Urea, CO(NH₂)₂, is found in the epidermis; moreover, urea may be synthesized in the laboratory. Urea plays a vital role in maintaining the skin's moisture balance and the suppleness of the skin. Urea is known to be a debriding agent, meaning urea may help in getting rid of the dead, damaged, or infected tissues. Urea is non-toxic, non-allergenic, colorless, and odorless. Additionally, urea may have anti-fungal and anti-microbial properties that may promote fast healing of dry cracked split skins and other types of skin problems.

Urea is naturally present in healthy skin, but when the skin is dry, and in some skin conditions the level of urea in the skin may be reduced. In the epidermis of healthy skin there is approximately 28 micrograms of urea per 2.5 square centimeters. In dry skin urea concentration may be diminished by about 50%. Urea can generally increase water content to the skin to a level of 97.8%.

Amount of micronized urea USP in disclosed urea silicone gel may be of about 8% by weight to about 25% by weight. Most suitable amount of micronized urea USP may be from about 15% by weight to about 20% by weight; depending on the skin condition to be treated.

In other embodiments, urea silicone gel may include a second with or without a third API to provide urea silicone gel with additional usage benefits. The second and third APIs may be a suitable pharmaceutical agent, herbal extract, and/or cosmetic agent, such as hydroxy-acids, among others.

According to an embodiment, urea may be combined with anhydrous silicone base in order to fabricate urea silicone gels. Anhydrous silicone base may include unique ingredients that may provide urea silicone gel potential healing and soothing power, emolliency and enhanced skin penetration.

Anhydrous Silicone Base

In one embodiment, urea silicone gel may include anhydrous silicone base that may improve the penetration of urea in skin as well as provide moisture and healing properties.

In addition, anhydrous silicone base may exfoliate dry scaly skin helping urea rapidly deliver skin softening, healing moisture deep into hard skin.

Ingredients within anhydrous silicone base may include Amazonian oils such as pracaxi oil and seje oil, which may be rich sources of essential fatty acids, behenic acid, oleic acid, and in some instances, lauric acid. The supply of essential fatty acids and antioxidant molecules may restore the cutaneous permeability and the function of the skin barrier. The supply of essential fatty acids and antioxidant molecules may also contribute to the control of the imperceptible water loss and maintain moisture of the skin.

In one embodiment, anhydrous silicone base may include a viscosity modulating agent, such as a gelling agent. Concentrations of viscosity modulating agent may be determined by one skilled in the art depending on the viscosity desired in order to obtain anhydrous silicone base that may be retained in the vicinity of the area of application for a brief period of time.

Amount of anhydrous silicone base that may be included in disclosed urea silicone gel may range from about 5% by weight to about 90% by weight; most suitable amount may be of about 10% by weight to about 25% by weight.

Pracaxi Oil

In one embodiment, disclosed urea silicone gel include a natural oil from the Amazon forest, named pracaxi oil which exhibits moisturizing properties as well as antimicrobial properties. Additionally, pracaxi oil may enhance the penetration of urea in skin, allowing a better absorption of micronized urea within urea silicone gel.

Pracaxi oil may be obtained from the seed oil of Pentaclethara Macroloba tree. Pracaxi oil may include about 20% behenic acid and about 35% oleic acid. In some cases, pracaxi oil may include more than these percentages. Behenic acid, oleic acid, and lauric acid, when used by themselves, may be irritating when applied to the skin. While having an irritating effect on the skin, behenic acid, oleic acid, and lauric acid, are also effective vehicles at delivering drugs, such as urea, through the skin. As the behenic acid and oleic acid are present within pracaxi oil, the effects of the acids may be less irritating on the skin, and as such makes pracaxi oil a good skin permeation enhancer. Pracaxi oil has been widely employed for its cosmetic, therapeutic, and medicinal properties. Pracaxi oil is rich in organic acids with antioxidant, antibacterial, antiviral, antiseptic, antifungal, anti-parasitic, and anti-hemorrhagic properties; therefore, pracaxi oil is suitable oil for some skin conditions healing treatment. Pracaxi oil may also aid lighten hyper-pigmentation caused by hormonal changes and as a direct result of skin conditions such as insect bites, abrasions, cuts, and acne. Additionally, pracaxi oil may improve the appearance of stretch marks.

Pracaxi may have a high amount of solid matter, not fatty acids, which make pracaxi oil solidify in cooler temperatures. The solid matter has gentle moisturizers and high cellular renewal properties, includes vitamin E and has essential fatty acids which make pracaxi oil suitable oil for scar treatment.

The fatty acid composition of pracaxi oil is illustrated below in Table 1.

TABLE 1
Fatty acid composition of pracaxi oil.

| Fatty Acidds | Carbon Atoms | Composition % |
|---|---|---|
| Lauric | 12:00 | 1.3000 |
| Myristic | 14:00 | 1.2100 |
| Palmitic | 16:00 | 2.0400 |
| Stearic | 18:00 | 2.1400 |
| Oleic | 18:10 | 44.3200 |
| Linoleic | 18:20 | 1.9600 |
| Linolenic | 18:30 | 2.3000 |
| Behenic | 22:00 | 19.6700 |
| Lignoceric | 24:00 | 14.8100 |

Amount of pracaxi oil within anhydrous silicone base may range from about 1% by weight to about 50% by weight; most suitable amount may be of about 1% by weight to about 15% by weight.

Seje Oil

Seje oil may be extracted from the mesocarp of the patauá palm and generally appears as a greenish-yellow and transparent liquid, with little odor and taste, having the physical appearance and composition of fatty acids that are similar to olive oil (Olea europaea). Seje oil has high content of unsaturated fatty acids. Seje oil has a high content of oleic acid therefore seje oil may be used as skin moisturizers. The dry mesocarp of patauá palm may include about 7.4% protein and possesses an excellent amino acid composition. Seje oil also includes a-tocopherol in its composition.

The fatty acid composition of seje oil is illustrated below in Table 2.

TABLE 2
Fatty acid composition of seje oil.

| Fatty Acids | Carbon Atoms | Composition % |
|---|---|---|
| Palmitic | 16:00 | 13.2 |
| Palmitoleic | 16:10 | — |
| Stearic | 18:00 | 3.6 |
| Oleic | 18:10 | 77.7 |
| Linoleic | 18:20 | 2.7 |
| Linolenic | 18:30 | 0.6 |
| Arachidic | 20:00 | 2 |
| Unsaturated | | 81.6 |

Amount of seje oil within anhydrous silicone base may range from about 15% by weight to about 25% by weight; most suitable amount may be of about 1% by weight to about 10% by weight.

In further embodiments other oils such as Plukenetia Volubilis seed oil, Inaja oil, Buriti, Tucuma, Bacuri, Ucuuba, Muru-Muru, and Copaiba, may be included in anhydrous silicone base within urea silicone gel.

Silicone

Anhydrous silicone base may include long chain silicone polymer (polysiloxanes), and silicone dioxide. Long chain silicone polymers cross link with silicone dioxide.

Silicone increases hydration of stratum corneum and therefore facilitates regulation of fibroblast production and reduction in collagen production resulting in softer and flatter scar.

Additionally, silicone within anhydrous silicone gel may protect the scarred tissue from bacterial invasion and may prevent bacteria-induced excessive collagen production in the scar tissue.

Furthermore, anhydrous silicone base may modulate the expression of growth factors, fibroblast growth factor β (FGF β) and tumor growth factor β (TGF β). TGF β stimulates fibroblasts to synthesize collagen and fibronectin. FGF β normalizes the collagen synthesis in an abnormal scar and increases the level of collagenases which breaks down the excess collagen, therefore balance of fibrogenesis and fibrolysis is ultimately restored.

Amount of silicone within anhydrous silicone base may range from about 5% by weight to about 85% by weight; most suitable amount may be of about 5% by weight to about 60% by weight.

Polyethylene Glycol (Peg) Ointment Base

In one embodiment, urea silicone gel may include a PEG ointment base that improves the penetration of urea in skin as well as providing moisture and healing properties.

PEG ointment base may promote a moist skin condition environment that may enhance the healing process. PEG ointment base is a soft, opaque ointment base that includes organic meadowsweet extract. Meadowsweet extract may include coumarins, flavonoids, rutin, heparin, mucilage, salicylic acid, and vitamin C, among others. Due to the phenolic glycosides (spiraein) and flavonoids in the meadowsweet extract, PEG ointment base may have germicidal, anti-inflammatory and healing properties, therefore PEG ointment base may be employed to treat sores, and cuts, among other skin conditions. Additionally, meadowsweet extract may have analgesic properties.

Amount of meadowsweet extract within PEG ointment base may range from about 0.1% by weight to about 2% by weight; most suitable amount may be of about 0.5% by weight to about 1% by weight.

PEG ointment base is a water-washable base for easy cleansing/debridement. PEG ointment base is adherent, may provide occlusion, and may maintain a moist environment at the skin condition/dressing interface. The objective of some skin conditions management is to provide conditions that will maintain a moist skin environment, which allows for optimal healing. Pain also may be decreased by maintaining a moist environment.

Disclosed urea silicone gel may include other beneficial agents and compounds such as acute or chronic moisturizing agents (including, for example, humectants, occlusive agents, and agents that affect the natural moisturization mechanisms of the skin), anti-oxidants, sunscreens having UVA and/or UVB protection, emollients, anti-irritants, pH adjusting agents, stabilizers, surfactants, gelling agents, vitamins, trace metals, anti-microbial agents, botanical extracts, fragrances, and/or dyes and color ingredients, among others.

Amount of PEG ointment base that may be included in disclosed urea silicone gel may range from about 1% by weight to about 95% by weight; most suitable amount may be of about 10% by weight to about 20% by weight.

Methods of Elaboration

Various methods may be used to produce disclosed urea silicone gel. In one embodiment, an electronic mixing system may be used employing a high shear force. Urea silicone gel may be mixed through number of different speed settings and time to achieve the desired particle size.

In one embodiment, in order to make urea silicone gel, anhydrous silicone base may be processed through an ointment mill to provide trituration, dispersion and reduce particle size of urea silicone gel. Anhydrous silicone base may be stirred under low shear conditions until a uniform formulation may be obtained. The urea silicone gel may be packed in suitable bottles or any suitable packaging.

Urea Silicone Gel Therapeutical Use and Application

Urea silicone gel may cause hard dry skin cells to “unpack” and expose their water binding sites, therefore enabling the cell to absorb and retain additional moisture. This action is also known as hydrotopic solubilization.

In an embodiment, the use of the combination of pracaxi oil and seje oil within urea silicone gel, may increase the skin permeability to urea, passing the stratum corneum and reaching the target area, particularly, because of the oil's high concentrations of oleic, linolenic, linoleic acids and sterols, particularly beta-sitosterol and stigmasterol.

In one embodiment, scar types that may be treated with disclosed urea silicone gel may include new scars, old scars, surgical scars, keloids, stretch marks, or skin conditions that would benefit from barrier protection.

In some embodiments, urea silicone gel may be used after surgery or an injury for reducing inflammation and to build up scar tissue; additionally, urea silicone gel may be applied on skin to soften and fade keloids and scar tissues.

In one embodiment, urea silicone gel may be applied on skin to treat excessive dryness that may be produced by the interaction of the stratum corneum with household or industrial chemicals or pollutants.

In another embodiment, urea silicone gel may be applied on skin grafts.

In one embodiment, urea silicone gel including about 8% by weight to about 10% by weight of urea, may be used as skin moisturizer in order to return water balance to skin therefore preventing and treating wrinkles.

In another aspect of the disclosure, urea silicone gel may be employed to prevent itching associated with scars or caused by insect bites and mild skin rashes.

In other embodiments disclosed urea silicone gel may be employed to treat skin spots, severe acne (and scars), stretch marks, and is useful for psoriasis and rosacea.

Increasing the permeability to urea, the time of treatment may be significantly reduced, therefore reducing the time of results of treatment to a period of from about 2 weeks to about 4 weeks.

In one embodiment, urea silicone gel may be applied to any area of skin such as face, heels, elbows, and joints, among others. Urea silicone gel may be topically applied in amount effective to increase the stratum corneum turnover rate of the skin.

According to an embodiment, urea silicone gel may be applied directly to the scar, twice daily (about 2 to 6 grams), during about 10 to 14 weeks, expecting results to show from about the second week after starting treatment.

In one aspect of the present disclosure, urea silicone gel may be administered without being covered by an adhesive bandage, patch or other physical barrier bonded to the administration area. In other embodiments, urea silicone gel may be administered being covered with an adhesive bandage, patch or other physical barrier bonded to the administration area.

Urea silicone gel may become touch dry within about one to three minutes of application to body surface.

It should be understood that the present disclosure is not limited in its application to the details of construction and arrangements of the components set forth here. The present disclosure is capable of other embodiments and of being practiced or carried out in various ways. Variations and modifications of the foregoing are within the scope of the present disclosure. It also being understood that the disclosure disclosed and defined here extends to all alternative combinations of two or more of the individual features mentioned or evident from the text. All of these different combinations constitute various alternative aspects of the present disclosure. The embodiments described here explain the best modes known for practicing the disclosure and will enable others skilled in the art to utilize the disclosure.

### EXAMPLES

Example #1 is an embodiment of formulation of urea silicone gels which may be employed for the treatment of stretch marks. Composition for example #1 urea silicone gel is described in table 3.

TABLE 3

| Ingredients | Percentages |
|---|---|
| Tretinoin | 0.05-0.1% |
| Urea Silicone Gel | qs 100 g |

Example #2 is an embodiment of formulation of urea silicone gels which may be employed for Psoriasis treatment. Composition for example #2 urea silicone gel is described in table 4.

TABLE 4

| Ingredients | Percentages |
|---|---|
| Zinc Pyrithionate | 0.25% |
| Clobetasol Propionate | 0.025-0.05% |
| Propylene Glycol | 2% |
| Urea Silicone Gel | qs 100 g |

Example #3 is an embodiment of formulation of urea silicone gels which may also be employed for Psoriasis treatment. Composition for example #3 urea silicone gel is described in table 5.

TABLE 5

| Ingredients | Percentages |
|---|---|
| Tacrolimus | 0.03-0.1% |
| Propylene Glycol | 2% |
| Urea Silicone Gel | qs 100 g |

Example #4 is an embodiment of formulation of urea silicone gels which includes corticoids, such as hydrocortisone. Composition for example #4 urea silicone gel is described in table 6.

TABLE 6

| Ingredients | Percentages |
|---|---|
| Hydrocortisone | 1-2% |
| Propylene Glycol | 2% |
| Urea Silicone Gel | qs 100 g |

Example #5 is an embodiment of formulation of urea silicone gels which includes corticoids, such as betamethasone. Composition for example #5 urea silicone gel is described in table 7.

TABLE 7

| Ingredients | Percentages |
|---|---|
| Betamethasone | 1-2% |
| Propylene Glycol | 2% |
| Urea Silicone Gel | qs 100 g |

Example #6 is an embodiment of formulation of urea silicone gels which includes anti-pruritic agents. Composition for example #6 urea silicone gel is described in table 8.

TABLE 8

| Ingredients | Percentages |
|---|---|
| Diphenhydramine HCL | 3% |
| Tripelennamine HCL | 2% |
| Hydrocortisone Acetate | 1% |
| Propylene Glycol | 2% |
| Ethyl Alcohol 190 Proof | 10% |
| Urea Silicone Gel | qs 100 g |

What is claimed is:

1. A method of treating a skin disorder, comprising applying to the skin disorder a pharmaceutical composition that comprises a silicon gel and urea.

3. The method according to claim 1, wherein said skin disorder is a keloid or hypertrophic scar.

4. The method according to claim 1, wherein said skin disorder is psoriasis or rosacea.

5. The method according to claim 1, wherein said skin disorder is hyperkeratosis.

6. The method according to claim 1, wherein the pharmaceutical composition further comprises an oil.

7. The method according to claim 6, wherein the oil is selected from the group consisting of pracaxi oil, seje oil, plukenetia volubilis oil, inaja oil, and combinations thereof.

8. The method according to claim 1, wherein the pharmaceutical composition further comprises one selected from the group consisting of anhydrous silicone, polyethylene glycol, and combinations thereof.

9. The method according to claim 8, wherein the pharmaceutical composition comprises about 5% to about 40% anhydrous silicone by weight.

10. The method according to claim 8, wherein the pharmaceutical composition comprises about 10% to about 25% anhydrous silicone by weight.

11. The method according to claim 8, wherein the pharmaceutical composition comprises about 1% to about 95% polyethylene glycol by weight.

12. The method according to claim 8, wherein the pharmaceutical composition comprises about 10% to about 20% polyethylene glycol by weight.

13. The method according to claim 1, wherein the urea comprises micronized urea.

14. The method according to claim 13, wherein the pharmaceutical composition comprises from about 8% to about 20% micronized urea by weight.

15. The method according to claim 13, wherein the pharmaceutical composition from about 15% to about 20% micronized urea by weight.

16. A method according to claim 1, wherein an effective amount of the pharmaceutical composition is about 2 to about 6 grams.

17. A composition, comprising pracaxi oil, seje oil, and urea.

18. A composition for use in treating stretch marks in humans comprising tretinoin and urea silicone gel.

19. The composition of claim 18, wherein the composition comprises about 0.05% to about 0.1% tretinoin by weight.

20. A composition for use in treating psoriasis in humans comprising zinc pyrithionate, clobetasol propionate, propylene glycol, and urea silicone gel.

21. The composition of claim 20, wherein the composition comprises about 0.25% zinc pyrithionate by weight, about 0.025% to about 0.05% clobetasol propionate by weight, and about 2.0% propylene glycol by weight.

22. A composition for use in treating psoriasis in humans comprising tacrolimus, propylene glycol, and urea silicone gel.

23. The composition of claim 22, wherein the composition comprises about 0.03% to about 0.1% tacrolimus by weight, and about 2.0% propylene glycol by weight.
